(19) United States
(12) Patent Application Publication
(10) Pub. No.: US 2010/0179432 A1
(43) Pub. Date: Jul. 15, 2010

(54) SYSTEMS AND METHODS FOR MAKING AND USING INTRAVASCULAR ULTRASOUND SYSTEMS WITH PHOTO-ACOUSTIC IMAGING CAPABILITIES

(75) Inventor: Peter Thornton, Los Altos, CA (US)

Correspondence Address: Boston Scientific Corporation;c/o Frommer Lawrence & Haug LLP, 745 Fifth Avenue, New York, NY, 10151, US

(73) Assignee: Boston Scientific SciMed, Inc., Maple Grove, MN (US)

(21) Appl. No.: 12/351,279

(22) Filed: Jan. 9, 2009

(51) Int. Cl. A61B 8/14

(52) U.S. Cl. 600/463; 600/466

(57) ABSTRACT

A catheter assembly for an intravascular ultrasound system includes a catheter with a lumen and an imaging core insertable into the lumen. The imaging core includes a rotatable driveshaft, at least one transducer, and at least one optical transport medium. The at least one transducer is mounted to the rotatable driveshaft and transforms applied electrical signals to acoustic signals and also transforms received acoustic signals to electrical signals. The at least one optical transport medium emits light and rotates with the driveshaft. The at least one transducer also receives acoustic signals generated by an object in response to illumination of the object by the light emitted from the distal end of the optical transport medium.

### TECHNICAL FIELD

The present invention is directed to the area of intravascular ultrasound imaging systems and methods of making and using the systems. The present invention is also directed to intravascular ultrasound systems that also include photo-acoustic imaging, as well as methods of making and using the intravascular ultrasound systems.

### BACKGROUND

Intravascular ultrasound (“IVUS”) imaging systems have proven diagnostic capabilities for a variety of diseases and disorders. For example, IVUS imaging systems have been used as an imaging modality for diagnosing blocked blood vessels and providing information to aid medical practitioners in selecting and placing stents and other devices to restore or increase blood flow. IVUS imaging systems have been used to diagnose atheromatous plaque build-up at particular locations within blood vessels. IVUS imaging systems can be used to determine the existence of an intravascular obstruction or stenosis, as well as the nature and degree of the obstruction or stenosis. IVUS imaging systems can be used to visualize segments of a vascular system that may be difficult to visualize using other intravascular imaging techniques, such as angiography, due to, for example, movement (e.g., a beating heart) or obstruction by one or more structures (e.g., one or more blood vessels not desired to be imaged). IVUS imaging systems can be used to monitor or assess ongoing intravascular treatments, such as angiography and stent placement in real (or almost real) time. Moreover, IVUS imaging systems can be used to monitor one or more heart chambers.

IVUS imaging systems have been developed to provide a diagnostic tool for visualizing a variety is diseases or disorders. An IVUS imaging system can include a control module (with a pulse generator, an image processor, and a monitor), a catheter, and one or more transducers disposed in the catheter. The transducer-containing catheter can be positioned in a lumen or cavity within, or in proximity to, a region to be imaged, such as a blood vessel wall or patient tissue in proximity to a blood vessel wall. The pulse generator in the control module generates electrical pulses that are delivered to the one or more transducers and transformed to acoustic pulses that are transmitted through patient tissue. Reflected pulses of the transmitted acoustic pulses are absorbed by the one or more transducers and transformed to electric pulses. The transformed electric pulses are delivered to the image processor and converted to an image displayable on the monitor.

Photo-acoustic imaging utilizes light and acoustic signals to form displayable images. In one exemplary photo-acoustic imaging technique, patient tissue is pulsed with light from a light source, such as a laser. Some of the emitted light is absorbed by the tissue and converted to heat. The heat causes a transient ultrasonic expansion of the illuminated tissue and a corresponding ultrasonic emission, which may be received by one or more transducers and processed into a displayable image.

### BRIEF SUMMARY

In one embodiment, a catheter assembly for an intravascular ultrasound system includes a catheter and an imaging core. The catheter has a distal end, a proximal end, and a longitudinal length. The catheter defines a lumen that extends along the longitudinal length of the catheter from the proximal end to the distal end. The imaging core is configured and arranged for inserting into the lumen. The imaging core includes a rotatable driveshaft, at least one transducer, at least one conductor, and at least one optical transport medium. The rotatable driveshaft has a distal end, a proximal end, and a longitudinal length. The at least one transducer is mounted to the distal end of the rotatable driveshaft and is configured and arranged for transforming applied electrical signals to acoustic signals and also for transforming received acoustic signals to electrical signals. The at least one conductor is coupled to the at least one transducer and extends along the longitudinal length of the driveshaft. The at least one optical transport medium has a proximal end, a distal end, and a longitudinal length. The distal end of the at least one optical transport medium is positioned in proximity to the at least one transducer. The at least one optical transport medium is configured and arranged to emit light from the distal end of the at least one optical transport medium and to rotate with the driveshaft. The at least one transducer is configured and arranged to receive acoustic signals generated by an object in response to illumination of the object by the light emitted from the distal end of the optical transport medium.

In another embodiment, an intravascular ultrasound imaging system includes a catheter, an imaging core, and a drive unit. The catheter has a distal end, a proximal end, and a longitudinal length. The catheter defines a lumen that extends along the longitudinal length of the catheter from the proximal end to the distal end. The imaging core is configured and arranged for inserting into the lumen. The imaging core includes a rotatable driveshaft, at least one transducer, at least one conductor, and at least one optical transport medium. The rotatable driveshaft has a distal end, a proximal end, and a longitudinal length. The at least one transducer is mounted to the distal end of the rotatable driveshaft and is configured and arranged for transforming applied electrical signals to acoustic signals and also for transforming received acoustic signals to electrical signals. The at least one conductor is coupled to the at least one transducer and extends along the longitudinal length of the driveshaft. The at least one optical transport medium has a proximal end, a distal end, and a longitudinal length. The distal end of the at least one optical transport medium is positioned in proximity to the at least one transducer. The at least one optical transport medium is configured and arranged to emit light from the distal end of the at least one optical transport medium and to rotate with the driveshaft. The at least one transducer is configured and arranged to receive acoustic signals generated by an object in response to illumination of the object by the light emitted from the distal end of the optical transport medium. The drive unit is coupled to the proximal end of the catheter. The drive unit includes a rotatable transformer and a motor. The rotatable transformer includes a rotor and a stator. The rotor is coupled to the proximal end of the driveshaft and defines at least one hollow shaft into which at least one optical transport medium is disposed. The at least one optical transport medium is configured and arranged to couple to the proximal end of the at least one optical transport medium disposed in the imaging core. The motor is for driving rotation of the driveshaft and is coupled to the rotatable transformer by a rotary motion interchanger.

In yet another embodiment, a method for photo-acoustic imaging of a patient using an intravascular ultrasound imaging system includes inserting a catheter into patient vasculature, illuminating patient tissue with light, receiving at least one emitted acoustic signal from the illuminated patient tissue, transmitting at least one acoustic signal to patient tissue from the at least one transducer, and receiving at least one reflected acoustic signal from the patient tissue. The catheter includes at least one optical transport medium coupled to a light source and at least one rotatable transducer electrically coupled to a control module by at least one conductor. The at least one optical transport medium rotates with the at least one transducer and maintains a constant position and direction relative to the at least one transducer. The light is emitted from the light source and is transmitted along the at least one optical transport medium.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an intravascular ultrasound imaging system, according to the invention;

FIG. 2 is a schematic side view of one embodiment of a catheter of an intravascular ultrasound imaging system, according to the invention;

FIG. 3 is a schematic perspective view of one embodiment of a distal end of the catheter shown in FIG. 2 with an imaging core disposed in a lumen defined in the catheter, according to the invention;

FIG. 4A is a schematic perspective view of one embodiment of a distal end of a catheter for an IVUS imaging system, the catheter including a light director for directing light emissions from the catheter and at least one transducer for transmitting and receiving acoustic signals, according to the invention;

FIG. 4B is a schematic longitudinal cross-sectional view of the distal end of the catheter shown in FIG. 4A, the catheter including an imaging core that includes an optical transport medium and a pair of conductors, according to the invention;

FIG. 5 is a schematic perspective view of one embodiment of a connector at a proximal end of the catheter shown in FIG. 4A, according to the invention;

FIG. 6 is a schematic cross-sectional view of one embodiment of a proximal end of the catheter shown in FIG. 4A coupled to a drive unit, according to the invention;

FIG. 7 is a schematic side view of one embodiment of the motor shown in FIG. 6 coupled to the transformer shown in FIG. 6 by a rotary motion interchanger, according to the invention;

FIG. 8 is a schematic distal perspective view of one embodiment of the motor shown in FIG. 6 coupled to the transformer shown in FIG. 6 by a rotary motion interchanger, according to the invention;

FIG. 9 is a schematic proximal perspective view of one embodiment of the motor shown in FIG. 6 coupled to the transformer shown in FIG. 6 by a rotary motion interchanger, according to the invention; and

FIG. 10 is a schematic longitudinal cross-sectional view of one embodiment of a light source configured and arranged to provide light to one or more optical transport media disposed in a proximal end of the transformer shown in FIG. 6 via one or more other optical transport media, according to the invention.

### DETAILED DESCRIPTION

The present invention is directed to the area of intravascular ultrasound imaging systems and methods of making and using the systems. The present invention is also directed to intravascular ultrasound systems that also include photo-acoustic imaging, as well as methods of making and using the intravascular ultrasound systems.

Suitable intravascular ultrasound (“IVUS”) imaging systems include, but are not limited to, one or more transducers disposed on a distal end of a catheter configured and arranged for percutaneous insertion into a patient. Examples of IVUS imaging systems with catheters are found in, for example, U.S. Pat. Nos. 7,306,561; and 6,945,938; as well as U.S. Patent Application Publication Nos. 20060253028; 20070016054; 20070038111; 20060173350; and 20060100522, all of which are incorporated by reference.

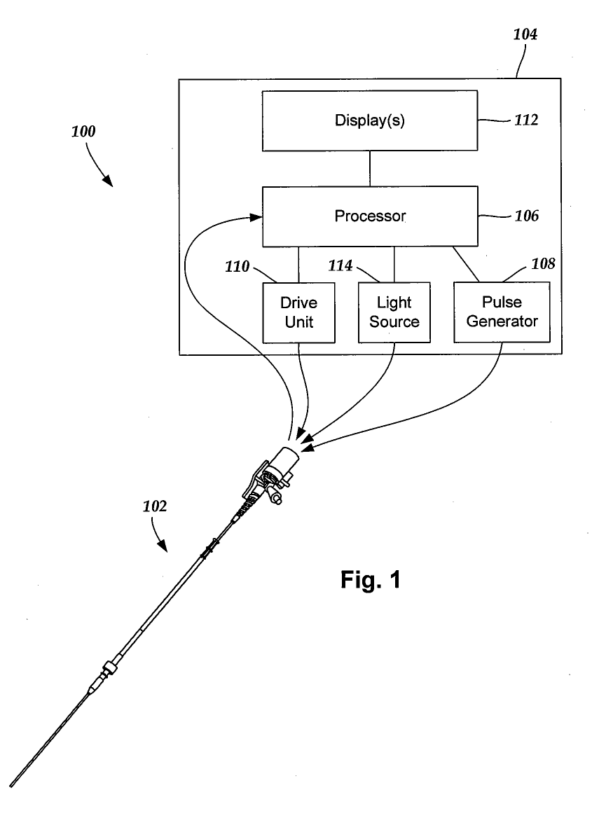

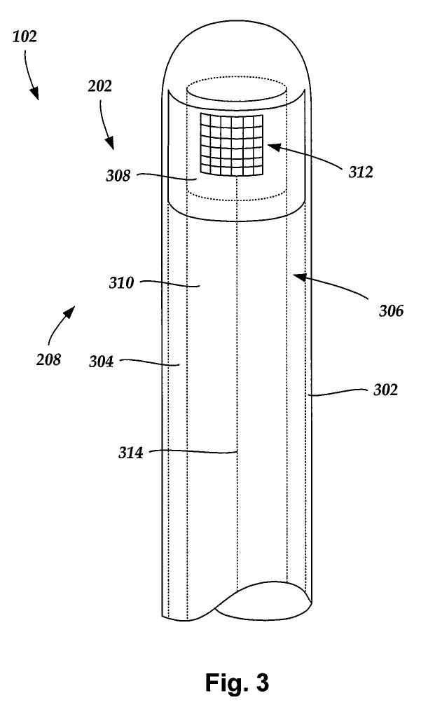

FIG. 1 illustrates schematically one embodiment of an IVUS imaging system 100. The IVUS imaging system 100 includes a catheter 102 that is coupleable to a control module 104. The control module 104 may include, for example, a processor 106, a pulse generator 108, a drive unit 110, and one or more displays 112. In at least some embodiments, the pulse generator 108 forms electric pulses that may be input to one or more transducers (312 in FIG. 3) disposed in the catheter 102. In at least some embodiments, mechanical energy from a motor disposed within the drive unit 110 may be used to drive an imaging core (306 in FIG. 3) disposed in the catheter 102. In at least some embodiments, the drive unit 110 additionally includes a transformer.

In at least some embodiments, electric pulses transmitted from the one or more transducers (312 in FIG. 3) may be input to the processor 106 for processing. In at least some embodiments, the processed electric pulses from the one or more transducers (312 in FIG. 3) may be displayed as one or more images on the one or more displays 112. In at least some embodiments, the processor 106 may also be used to control the functioning of one or more of the other components of the control module 104. For example, the processor 106 may be used to control at least one of the frequency or duration of the electrical pulses transmitted from the pulse generator 108, the rotation rate of the imaging core (306 in FIG. 3) by the drive unit 110, the velocity or length of the pullback of the imaging core (306 in FIG. 3) by the drive unit 110, or one or more properties of one or more images formed on the one or more displays 112. As discussed in more detail below, a light source 114 is provided to generate light for illuminating patient tissue in proximity to a distal end of the catheter 102.

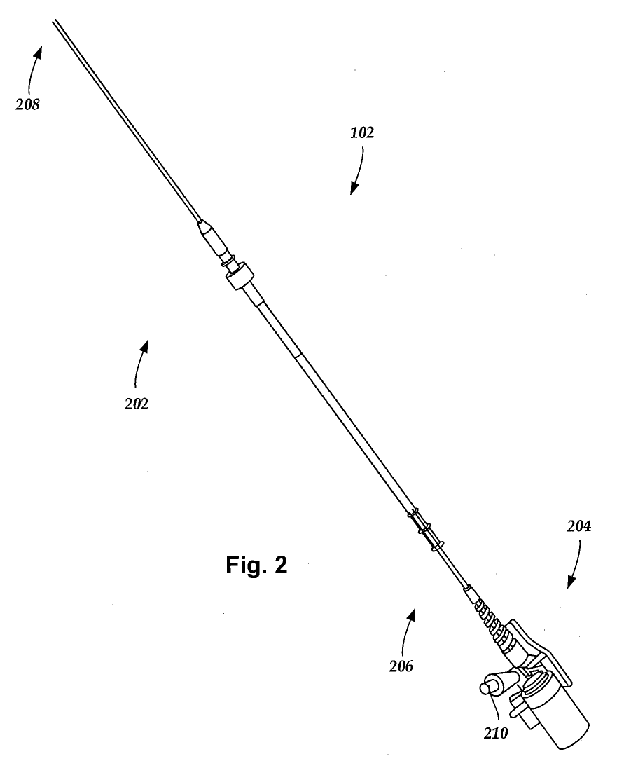

FIG. 2 is a schematic side view of one embodiment of the catheter 102 of the IVUS imaging system (100 in FIG. 1). The catheter 102 includes an elongated member 202 and a hub 204. The elongated member 202 includes a proximal end 206 and a distal end 208. In FIG. 2, the proximal end 206 of the elongated member 202 is coupled to the catheter hub 204 and the distal end 208 of the elongated member is configured and arranged for percutaneous insertion into a patient. In at least some embodiments, the catheter 102 defines at least one flush port, such as flush port 210. In at least some embodiments, the flush port 210 is defined in the hub 204. In at least some embodiments, the hub 204 is configured and arranged to couple to the control module (104 in FIG. 1). In some embodiments, the elongated member 202 and the hub 204 are formed as a unitary body. In other embodiments, the elongated member 202 and the catheter hub 204 are formed separately and subsequently assembled together.

FIG. 3 is a schematic perspective view of one embodiment of the distal end 208 of the elongated member 202 of the catheter 102. The elongated member 202 includes a sheath 302 and a lumen 304. An imaging core 306 is disposed in the lumen 304. The imaging core 306 includes an imaging device 308 coupled to a distal end of a rotatable driveshaft 310.

The sheath 302 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

One or more transducers 312 may be mounted to the imaging device 308 and employed to transmit and receive acoustic pulses. In a preferred embodiment (as shown in FIG. 3), an array of transducers 312 are mounted to the imaging device 308. In other embodiments, a single transducer may be employed. In yet other embodiments, multiple transducers in an irregular-array may be employed. Any number of transducers 312 can be used. For example, there can be two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used.

The one or more transducers 312 may be formed from one or more known materials or devices capable of transforming applied electrical pulses to pressure distortions on the surface of the one or more transducers 312, and vice versa. Examples of suitable materials or devices include piezoelectric ceramic materials, piezocomposite materials, piezoelectric plastics, barium titanates, lead zirconate titanates, lead metaniobates, polyvinylidenefluorides, capacitive micromachined ultrasonic transducers, and the like.

The pressure distortions on the surface of the one or more transducers 312 form acoustic pulses of a frequency based on the resonant frequencies of the one or more transducers 312. The resonant frequencies of the one or more transducers 312 may be affected by the size, shape, and material used to form the one or more transducers 312. The one or more transducers 312 may be formed in any shape suitable for positioning within the catheter 102 and for propagating acoustic pulses of a desired frequency in one or more selected directions. For example, transducers may be disc-shaped, block-shaped, rectangular-shaped, oval-shaped, and the like. The one or more transducers may be formed in the desired shape by any process including, for example, dicing, dice and fill, machining, microfabrication, and the like.

As an example, each of the one or more transducers 312 may include a layer of piezoelectric material sandwiched between a conductive acoustic lens and a conductive backing material formed from an acoustically absorbent material (e.g., an epoxy substrate with tungsten particles). During operation, the piezoelectric layer may be electrically excited by both the backing material and the acoustic lens to cause the emission of acoustic pulses.

In at least some embodiments, the one or more transducers 312 can be used to form a radial cross-sectional image of a surrounding space. Thus, for example, when the one or more transducers 312 are disposed in the catheter 102 and inserted into a blood vessel of a patient, the one more transducers 312 may be used to form an image of the walls of the blood vessel and tissue surrounding the blood vessel.

The imaging core 306 is rotated about a longitudinal axis of the catheter 102. As the imaging core 306 rotates, the one or more transducers 312 emit acoustic pulses in different radial directions. When an emitted acoustic pulse with sufficient energy encounters one or more medium boundaries, such as one or more tissue boundaries, a portion of the emitted acoustic pulse is reflected back to the emitting transducer as an echo pulse. Each echo pulse that reaches a transducer with sufficient energy to be detected is transformed to an electrical signal in the receiving transducer. The one or more transformed electrical signals are transmitted to the control module (104 in FIG. 1) where the processor 106 processes the electrical-signal characteristics to form a displayable image of the imaged region based, at least in part, on a collection of information from each of the acoustic pulses transmitted and the echo pulses received.

As the one or more transducers 312 rotate about the longitudinal axis of the catheter 102 emitting acoustic pulses, a plurality of images are formed that collectively form a radial cross-sectional image of a portion of the region surrounding the one or more transducers 312, such as the walls of a blood vessel of interest and the tissue surrounding the blood vessel. In at least some embodiments, the radial cross-sectional image can be displayed on one or more displays 112.

In at least some embodiments, the imaging core 306 may move longitudinally within the lumen of the catheter 102 while the catheter 102 remains stationary. For example, the imaging core 306 may be advanced (moved towards the distal end of the catheter 102) or retracted/pulled back (moved towards the proximal end of the catheter 102) within the lumen 304 of the catheter 102 while the catheter 102 remains in a fixed location within patient vasculature (e.g., blood vessels, the heart, and the like). During longitudinal movement (e.g., pullback) of the imaging core 306, an imaging procedure may be performed, wherein a plurality of cross-sectional images are formed along a longitudinal length of patient vasculature.

In at least some embodiments, the catheter 102 includes at least one retractable section that can be retracted during an imaging procedure. In at least some embodiments, a motor disposed in the drive unit (110 in FIG. 1) drives the pullback of the imaging core 306 within the catheter 102. In at least some embodiments, the pullback distance of the imaging core is at least 5 cm. In at least some embodiments, the pullback distance of the imaging core is at least 10 cm. In at least some embodiments, the pullback distance of the imaging core is at least 15 cm. In at least some embodiments, the pullback distance of the imaging core is at least 20 cm. In at least some embodiments, the pullback distance of the imaging core is at least 25 cm.

The quality of an image produced at different depths from the one or more transducers 312 may be affected by one or more factors including, for example, bandwidth, transducer focus, beam pattern, as well as the frequency of the acoustic pulse. The frequency of the acoustic pulse output from the one or more transducers 312 may also affect the penetration depth of the acoustic pulse output from the one or more transducers 312. In general, as the frequency of an acoustic pulse is lowered, the depth of the penetration of the acoustic pulse within patient tissue increases. In at least some embodiments, the IVUS imaging system 100 operates within a frequency range of 5 MHz to 60 MHz.

In at least some embodiments, one or more conductors 314 electrically couple the transducers 312 to the control module (104 in FIG. 1). In at least some embodiments, the one or more conductors 314 extend along a longitudinal length of the imaging core 306. In at least some embodiments, the one or more conductors 314 may extend along at least a portion of the longitudinal length of the catheter 102 as shielded electrical cables, such as a coaxial cable, or a twisted pair cable, or the like.

In at least some embodiments, the catheter 102 with one or more transducers 312 mounted to the distal end 208 of the imaging core 306 may be inserted percutaneously into a patient via an accessible blood vessel, such as the femoral artery, at a site remote from the selected portion of the selected region, such as a blood vessel, to be imaged. The catheter 102 may then be advanced through the blood vessels of the patient to the selected imaging site, such as a portion of a selected blood vessel.

Differentiating between two or more different tissue types displayed on an IVUS image is desirable, but can be difficult using the IVUS image. For example, it may be difficult to determine where a border between two or more tissue types is located, or even if a border exists.

One technique for tissue differentiation is photo-acoustic imaging, wherein patient tissue is pulsed with light from a light source, such as a laser. When patient tissue is pulsed with light, some of the emitted light is absorbed by the tissue and converted to heat. The heat causes a transient ultrasonic expansion of the illuminated tissue and a corresponding ultrasonic emission, which may be received by one or more transducers and processed into a displayable image (or one or more audible tones).

Photo-acoustic imaging capabilities may be incorporated into an IVUS imaging system. Such an arrangement includes a light source that provides light to an optical transport medium (such as an optical fiber) to transport light to a distal portion of the catheter. Light is emitted from the distal end of the optical transport medium in proximity to one or more transducers disposed on an imaging core in the catheter so that the subsequently-emitted acoustic pulses from the illuminated tissue may be received by the one or more transducers.

The one or more transducers disposed on the imaging core rotate. Thus, it is desirable to have the light-emitting portion of the optical transport medium also be disposed on the imaging core so that the light-emitting portion of the optical transport medium also rotates, thereby maintaining a constant relative position with respect to the one or more transducers. However, the proximal portion of an imaging core is generally obstructed by the drive unit (which, as discussed above, includes the transformer and the motor). Thus, there is no convenient way to provide light from a light source to the optical transport medium.

Previous systems have embedded optical fibers in a sheath of a catheter. However, embedding optical fibers in the sheath can make sheath manufacturing difficult. Moreover, the embedded optical fibers do not rotate with transducers. Additionally, embedding optical fibers in the sheath may hinder, or even eliminate, the pullback function of the imaging core during an imaging procedure.

In at least some embodiments, an IVUS imaging system incorporates photo-acoustic imaging capabilities into the IVUS imaging system. In at least some embodiments, one or more optical transport media (e.g., optical fibers, light pipes, light guides, light tubes, or the like) are disposed in an imaging core of the IVUS imaging system. In at least some embodiments, the one or more optical transport media disposed in the imaging core couple to additional optical transport media disposed in a transformer of a drive unit of the IVUS imaging system. In at least some embodiments, a proximal end of the transformer provides access to the one or more optical transport media disposed in a rotor of the transformer, thereby allowing the light source to provide light to the optical transport medium. In at least some embodiments, the transformer is positioned side-by-side with the motor and is coupled to the motor via one or more rotary motion interchangers (e.g., a timing belt, one or more gears, and the like).

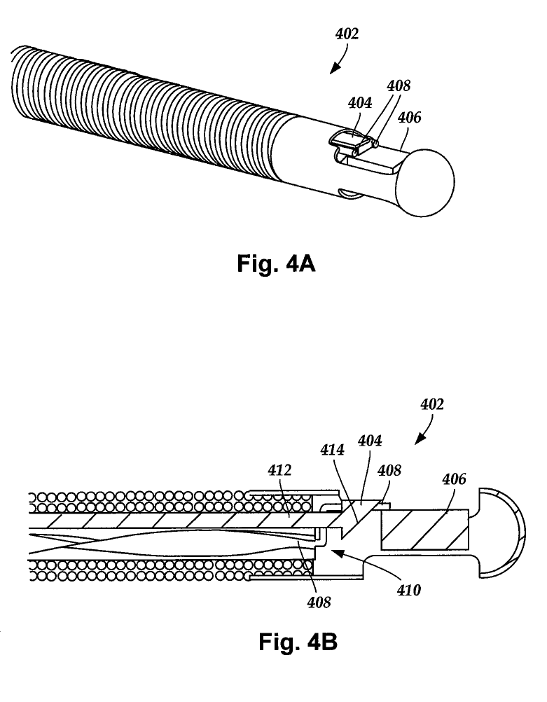

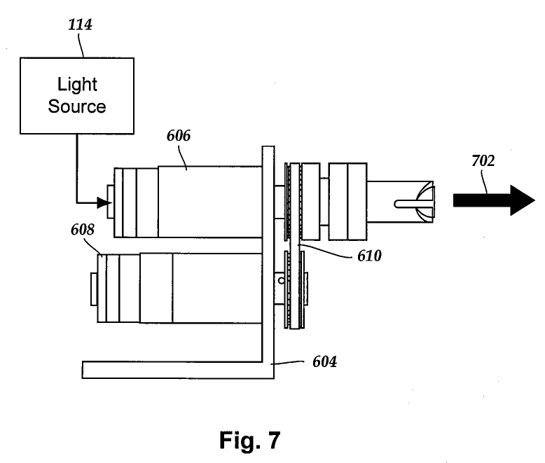

FIG. 4A is a schematic perspective view of one embodiment of a distal end of a catheter 402 for an IVUS imaging system (100 in FIG. 1). The catheter 402 includes a light director 404 and one or more transducers 406. The one or more transducers 406 are coupled to the processor (106 in FIG. 1) via one or more conductors 408 disposed in an imaging core (410 in FIG. 4B). In at least some embodiments, the light director 404 may be used to direct light that is emitted from a light source (704 in FIG. 7) and transported along a longitudinal length of the catheter 402 via one or more optical transport media (412 in FIG. 4B).

In at least some embodiments, the light director 404 includes a mirror (414 in FIG. 4B) to redirect the light from an optical transport medium to a desired tissue. In at least some embodiments, the light director 404 includes a diffuser to diffuse light from a narrow point source (such as an optical transport medium). In at least some embodiments, the light director 404 includes a mirror (414 in FIG. 4B) and a diffuser. In at least some embodiments, the mirror (414 in FIG. 4B) and the diffuser are separate from one another. In at least some embodiments, the diffuser is on the emitting end of the optical transport medium. In at least some embodiments, the mirror (414 in FIG. 4B) has a light-diffusing reflective surface. The light director 404 may be fabricated from any material suitable for reflecting or orienting light including, for example, glass, plastic, and the like or combinations thereof.

FIG. 4B is a schematic longitudinal cross-sectional view of one embodiment of a distal end of the catheter 402. The catheter 402 includes a lumen into which an imaging core 410 is disposed. In at least some embodiments, the imaging core 410 includes the one or more conductors 408 extending along at least a portion of the imaging core 410. The imaging core 410 also includes one or more optical transport media, such as optical transport medium 412, also extending along at least a portion of the imaging core 410. In at least some embodiments, the one or more conductors 408 may extend along at least a portion of the longitudinal length of the catheter 402 as shielded electrical cables, such as a coaxial cable, or a twisted pair cable, or the like. In at least some embodiments, the one or more optical transport media 412 wrap at least one time around the one or more conductors 408. In at least some embodiments, the one or more optical transport media 412 are coupled at a distal end to the light director 404, through which light from the one or more optical transport media 412 is emitted. In at least some embodiments, the light director 404 and the one or more transducers 406 have the same rotational velocity. In at least some embodiments, the light director 404 maintains a constant relative position with respect to the one or more transducers 406. In at least some embodiments, the light director 404 is fixed to the one or more transducers 406.

Light provided from a light source (704 in FIG. 7) may be transported along one or more optical transport media 412, passed through the light director 404, and used to illuminate selected patient tissue for photo-acoustic imaging. In at least some embodiments, the light may be emitted in one or more timed patterns, such as pulses. In at least some embodiments, the light emission may be timed with respect to the transmission of one or more acoustic signals from the one or more transducers 406.

In at least some embodiments, the IVUS imaging system may be used to perform photo-acoustic imaging without performing ultrasound imaging. In at least some embodiments, the IVUS imaging system is configured to perform both photo-acoustic imaging and ultrasound imaging, either sequentially or independently. In at least some embodiment, the data from a photo-acoustic image and an ultrasound image may be combined to form a composite image.

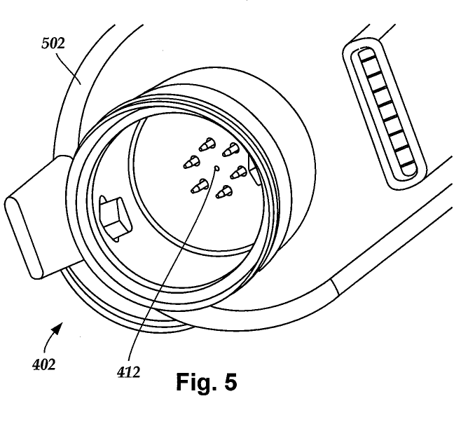

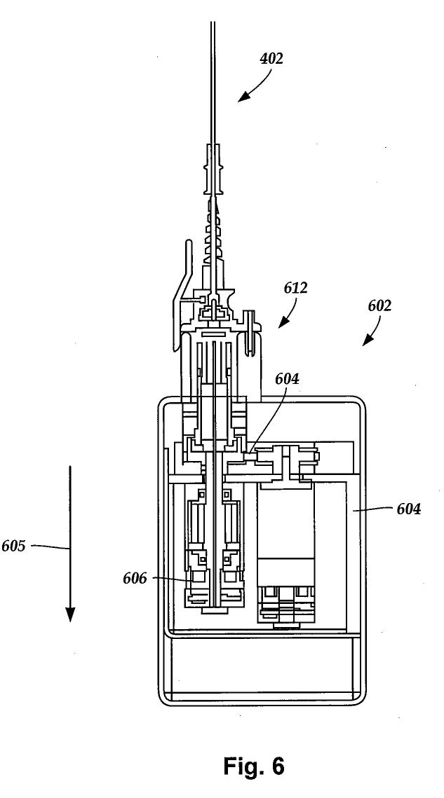

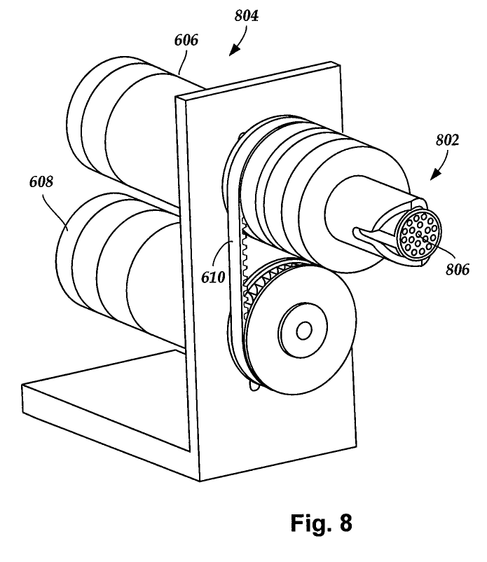

In at least some embodiments, a proximal end of the catheter 402 couples with a transformer (606 in FIG. 6) disposed in a drive unit (602 in FIG. 6). FIG. 5 is a schematic perspective view of one embodiment of a connector 502 in a proximal end of the catheter 402. In at least some embodiments, the connector 502 is configured and arranged to couple to a drive unit (602 in FIG. 6). A proximal end of the one or more optical transport media 412 terminates in proximity to the connector 502. In at least some embodiments, a coupler (not shown in FIG. 5) may be disposed at the proximal end of the catheter 402 to facilitate coupling of the one or more optical transport media 412 to one or more optical transport media (806 in FIG. 8) disposed in the transformer (606 in FIG. 6).

FIG. 6 is a schematic cross-sectional view of one embodiment of a proximal end of the catheter 402 coupled to a drive unit 602. The drive unit 602 includes a drive sled 604 configured and arranged to slide along a length of the drive unit 602 during pullback of the imaging core (410 in FIG. 4B) in the direction shown by directional arrow 605, thereby retracting the proximal end of the catheter 402. The drive unit 602 also includes a rotary transformer 606 and a rotary motor 608. In FIG. 6, the rotary transformer 606 and the rotary motor 608 are shown coupled to the drive sled 604.

The transformer 606 and the motor 608 are coupled to one another via a rotary motion interchanger 610. In at least some embodiments, the motor 608 drives the rotation of the transformer 606 which, in turn, drives the rotation of the imaging core (410 in FIG. 4B). Thus, coupling the transformer 606 to the motor 608 via the rotary motion interchanger 610 permits the transformer 606 and the motor 608 to be coupled together without a proximal end of the transformer 606 being obstructed by the motor 608, thereby providing an access point for light from a light source (704 in FIG. 7) into the imaging core (410 in FIG. 4B).

In at least some embodiments, positioning the transformer 606 and the motor 608 adjacent to one another and coupled via the rotary motion interchanger 610 may allow the size of the drive unit 602 to be reduced. In at least some embodiments, gearing may be added to the drive train to increase the torque delivered by the motor 608. In at least some embodiments, positioning the transformer 606 and the motor 608 adjacent to one another may also allow the motor 608 to operate at a more efficient velocity range, thereby decreasing the temperature of the motor 608. In at least some embodiments, the motor and the transformer have the same rotational velocity. In at least some embodiments, gearing may be added so that the transformer has a rotational velocity that is different from the rotational velocity of the motor.

The transformer 606 is also coupled to the proximal end of the catheter 402. In at least some embodiments, the catheter 402 is coupled to the transformer 606 via a drive unit connector 612 which couples to the connector (502 in FIG. 5) disposed at the proximal end of the catheter 402. In at least some embodiments, the one or more optical transport media (412 in FIG. 4B) disposed in the imaging core (410 in FIG. 4B) couple to one or more optical transport media (806 in FIG. 8) disposed in the transformer 606.

In at least some embodiments, the transformer 606 and the motor 608 are positioned such that the one or more optical transport media disposed in the transformer 606 are accessible at both a proximal and a distal end of the transformer 606. FIG. 7 is a schematic side view of one embodiment of the transformer 606 and the motor 608 coupled to the drive sled 604 and to the rotary motion interchanger 610. A distal end of the transformer 606 is configured and arranged to couple with the catheter (402 in FIG. 6), as shown by directional arrow 702. A proximal end of the transformer 606 is configured and arranged to provide access to one or more optical transport media disposed in the transformer 606 by a light source 114.

In at least some embodiments, the light source 114 is disposed in the control module (104 in FIG. 1). In at least some embodiments, the light source 114 is a laser. In at least some embodiments, the light from the light source 114 may be emitted at various frequencies. Different types of tissues may respond differently to being illuminated by light of different frequencies. Thus, illuminating tissue with various frequencies of light and processing received acoustic signals from the illuminated tissue may be used as a way to differentiate between two or more types of tissues.

FIG. 8 is a schematic proximal perspective view of one embodiment of a distal end of the transformer 606 being coupled to the motor 608 via the rotary motion interchanger 610. The transformer includes a rotor 802 and a stator 804. The one or more optical transport media 806 terminate in proximity to the distal end of the transformer 606. In at least some embodiments, the one or more optical transport media 804 disposed in the transformer are configured and arranged to couple to the one or more optical transport media (412 in FIG. 4B) disposed in the catheter (402 in FIG. 4A). In at least some embodiments, a coupler (not shown in FIG. 8) may be disposed at the distal end of the transformer 606 to facilitate coupling of one or more optical transport media 806 disposed in the transformer 606 to the one or more optical transport media (412 in FIG. 4B) disposed in the catheter (402 in FIG. 4B).

In at least some embodiments, the one or more optical transport media disposed in the transformer 606 extend along one or more hollow shafts defined in the rotor 802 of the transformer 606. In at least some embodiments, the one or more optical transport media 806 (e.g., optical fibers, light pipes, light guides, light tubes, and the like) are the same type of optical transport medium as the one or more optical transport media (412 in FIG. 4B). In at least some embodiments, the one or more optical transport media 806 are a different type of optical transport medium from the one or more optical transport media (412 in FIG. 4B). For example, in at least one embodiment, the one or more optical transport media (412 in FIG. 4B) may be one or more optical fibers and the one or more optical transport medium 806 may be one or more light pipes. In at least one other embodiment, the one or more optical transport media (412 in FIG. 4B) may be one or more light pipes and the one or more optical transport media 806 may be one or more optical fibers. In at least some embodiments, more than one type of optical transport medium may be used in combination. For example, the one or more optical transport media 806 may include one or more optical fibers and one or more light pipes.

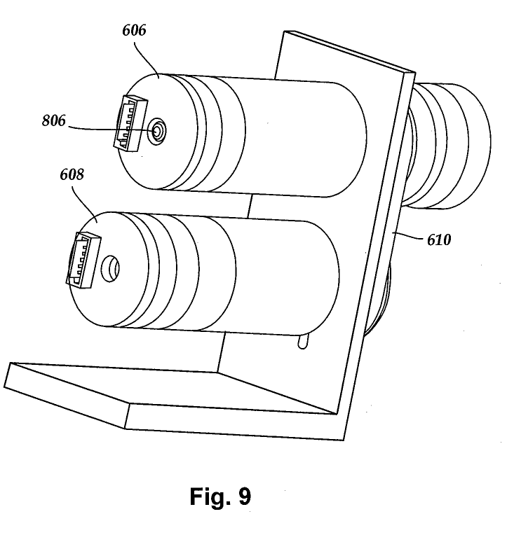

FIG. 9 is a schematic proximal perspective view of one embodiment of a distal end of the transformer 606 being coupled to the motor 608 via the rotary motion interchanger 610. In FIG. 9, the one or more optical transport media 806 are shown terminating in proximity to the proximal end of the transformer 606. In at least some embodiments, one or more additional materials may be employed to facilitate the emission of light at the interfaces between optical transport media (i.e., between the one or more optical transport media 412 disposed in the catheter 402 and the one or more optical transport media 806 disposed in the transformer 606, and between the one or more optical transport media 806 disposed in the transformer 606 and the light source 114) including, for example, one or more types of optical grease.

In at least some embodiments, the one or more optical transport media 806 are positioned in the center of the rotor such that there is little, if any, wobble as the one or more optical transport media 806 rotate with the rotor. In at least some embodiments, the one or more optical transport media 806 are positioned off center of the rotor. In at least some embodiments, the light source may be positioned in proximity to the proximal portion of the transformer. In at least some embodiments, the light source emits light directly into a proximal end of the one or more optical transport media 806. In at least some other embodiments, the light source provides light to the one or more optical transport media 806 via one or more optical transport media.

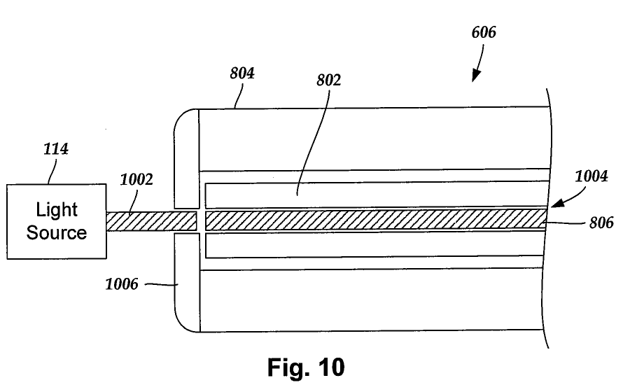

FIG. 10 is a schematic longitudinal cross-sectional view of one embodiment of the light source 114 configured and arranged to provide light to one or more optical transport media 806 disposed in a proximal end of the transformer 606 via one or more optical transport media 1002. In FIG. 10, the transformer 606 includes the rotor 802 and the stator 804. The one or more optical transport media 806 are disposed in a hollow shaft 1004 defined in the rotor 802. In at least some embodiments, a coupler 1006 may be disposed at the proximal end of the transformer 606 and configured and arranged to hold the one or more optical transport media 1002 in a steady position in proximity to the proximal end of the one or more optical transport media 806. In at least some embodiments, the coupler 1006 is an end cap with an aperture through which the one or more optical transport media 1002 extend.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A catheter assembly for an intravascular ultrasound system, the catheter assembly comprising:
a catheter having a distal end, a proximal end, and a longitudinal length, the catheter defining a lumen extending along the longitudinal length of the catheter from the proximal end to the distal end; and
an imaging core configured and arranged for inserting into the lumen, the imaging core comprising
a rotatable driveshaft having a distal end, a proximal end, and a longitudinal length,
at least one transducer mounted to the distal end of the rotatable driveshaft, the at least one transducer configured and arranged for transforming applied electrical signals to acoustic signals and also for transforming received acoustic signals to electrical signals,
at least one conductor coupled to the at least one transducer, the at least one conductor extending along the longitudinal length of the driveshaft, and
at least one optical transport medium having a proximal end, a distal end, and a longitudinal length, the distal end of the at least one optical transport medium positioned in proximity to the at least one transducer, the at least one optical transport medium configured and arranged to emit light from the distal end of the at least one optical transport medium and to rotate with the driveshaft, wherein the at least one transducer is configured and arranged to receive acoustic signals generated by an object in response to illumination of the object by the light emitted from the distal end of the optical transport medium.

2. The catheter assembly of claim 1, wherein the imaging core further comprises at least one light director configured and arranged for directing light emitted from the distal end of the at least one optical transport medium.

3. The catheter assembly of claim 2, wherein the at least one light director is at least one of a mirror or a diffuser.

4. The catheter assembly of claim 1, wherein the at least one optical transport medium comprises at least one of an optical fiber, a light pipe, a light guide, or a light tube.

5. The catheter assembly of claim 1, wherein the at least one conductor comprises a twisted pair of conductive wires, wherein the at least one optical transport medium wraps at least one time around the twisted pair of conductive wires.

6. The catheter assembly of claim 1, further comprising a light source coupled to the proximal end of the at least on optical transport medium, the light source configured and arranged to generate pulses of light at one or more selected frequencies.

7. An intravascular ultrasound imaging system comprising:
a catheter having a distal end, a proximal end, and a longitudinal length, the catheter defining a lumen extending along the longitudinal length of the catheter from the proximal end to the distal end;
an imaging core configured and arranged for inserting into the lumen, the imaging core comprising
a rotatable driveshaft having a distal end, a proximal end, and a longitudinal length,
at least one transducer mounted to the distal end of the rotatable driveshaft, the at least one transducer configured and arranged for transforming applied electrical signals to acoustic signals and also for transforming received echo signals to electrical signals,
at least one conductor coupled to the at least one transducer, the at least one conductor extending along the longitudinal length of the driveshaft, and
at least one optical transport medium having a proximal end, a distal end, and a longitudinal length, the distal end of the at least one optical transport medium positioned in proximity to the at least one transducer, the at least one optical transport medium configured and arranged to emit light from the distal end of the at least one optical transport medium and to rotate with the driveshaft, wherein the at least one transducer is configured and arranged to receive acoustic signals generated by an object in response to illumination of the object by the light emitted from the distal end of the optical transport medium; and
a drive unit coupled to the proximal end of the catheter, the drive unit comprising
a rotatable transformer comprising a rotor and a stator, wherein the rotor is coupled to the proximal end of the driveshaft, the rotor defining at least one hollow shaft into which at least one optical transport medium is disposed, the at least one optical transport medium disposed in the transformer configured and arranged to couple to the proximal end of the at least one optical transport medium disposed in the imaging core, and
a motor for driving rotation of the driveshaft, the motor coupled to the rotatable transformer by a rotary motion interchanger.

8. The system of claim 7, further comprising a control module coupled to the imaging core, the control module comprising
a pulse generator configured and arranged for providing electric signals to the at least one transducer, the pulse generator electrically coupled to the at least one transducer via the one or more conductors and the rotatable transformer, and
a processor configured and arranged for processing received electrical signals from the at least one transducer to form at least one image, the processor electrically coupled to the at least one transducer via the one or more conductors and the rotatable transformer.

9. The system of claim 8, further comprising at least one coupler for facilitating coupling of at least one of: 1) the at least one optical transport medium disposed in the imaging core to the at least one optical transport medium disposed in the transformer; or 2) the at least one optical transport medium disposed in the transformer to the light source.

10. The system of claim 8, further comprising optical grease disposed at the interface between at least one of 1) the at least one optical transport medium disposed in the imaging core and the at least one optical transport medium disposed in the transformer; or 2) the at least one optical transport medium disposed in the transformer and the light source.

11. The system of claim 8, wherein the control module further comprises at least one display electrically coupled to the processor, the at least one display configured and arranged for displaying the at least one image formed by the processor.

12. The system of claim 7, wherein the rotary motion interchanger comprises at least one of a timing belt or at least one gear.

13. The system of claim 7, wherein the drive unit further comprises a drive sled configured and arranged for controlling longitudinal movement of the imaging core within the lumen of the catheter.

14. The system of claim 10, wherein the transformer and the motor are coupled to the drive sled.

15. The system of claim 7, further comprising a light source configured and arranged to provide light to the at least one optical transport medium disposed in the transformer, the at least one light source configured and arranged to generate pulses of light.

16. The system of claim 15, wherein the light source is a laser.

17. A method for photo-acoustic imaging of a patient using an intravascular ultrasound imaging system, the method comprising:
inserting a catheter into patient vasculature, the catheter comprising at least one optical transport medium coupled to a light source and at least one rotatable transducer electrically coupled to a control module by at least one conductor, wherein the at least one optical transport medium rotates with the at least one transducer and maintains a constant position and direction relative to the at least one transducer;
illuminating patient tissue with light emitted from the light source and transmitted along the at least one optical transport medium;
receiving at least one emitted acoustic signal from the illuminated patient tissue;
transmitting at least one acoustic signal to patient tissue from the at least one transducer; and
receiving at least one reflected acoustic signal from the patient tissue.

18. The method of claim 17, wherein the received at least one emitted acoustic signal and the received at least one reflected acoustic signal are transmitted to a processor for processing.

19. The method of claim 18, further comprising displaying an image based on the received and processed at least one emitted acoustic signal and the received and processed at least one reflected acoustic signal.

20. The method of claim 17, wherein the at least one optical transport medium and the at least one conductor are disposed in a rotatable imaging core disposed in the catheter.
